(19) United States
(12) Patent Application Publication
(10) Pub. No.: US 2011/0092507 A1
(43) Pub. Date: Apr. 21, 2011

(54) Process for preparing R-(+)-3-morpholino-4-(3- tert-butylamino-2-hydroxypropoxy)-1,2,5-thiadiazole

(76) Inventors: Chin-Tsai FAN, Sinying City (TW); Chen-Ming Hsiao, Sinying City (TW); Rong-Bin Hsieh, Sinying City (TW)

(21) Appl. No.: 12/581,987

(22) Filed: Oct. 20, 2009

(51) Int. Cl. A61K 31/5377; C07D 417/04

(52) U.S. Cl. 514/236.2; 544/134

(57) ABSTRACT

The present invention provides a process for preparing optically active timolol. The process comprises the following steps. Firstly, reacting 3-hydroxy-4-morpholino-1,2,5-thiadiazole with an optically active epichlorohydrin in the presence of a solvent system, which has a first volume and a catalyst optionally in the presence of a suitable base to obtain an optically active intermediate product. Secondly, treating the optically active intermediate product with a solution, which has a second volume and comprises tert-butylamine to obtain an optically active timolol. The solvent system used in the first step can be an amide solvent, sulfoxide solvent, cyclic hydrocarbon solvent, ketone solvent, or a heterocyclic solvent. The catalyst used in the first step can be an alkali metal hydroxide, alkali metal carbonate, alkali metal hydrogen carbonate, piperidine, pyridine, triethylamine, potassium hydroxide, sodium hydroxide, potassium carbonate, and other heterocyclic bases.

### BACKGROUND OF THE INVENTION

1. Technical Field

The present invention relates to a process of preparing an optically active compound; and more particularly, to a process of preparing an optically active timolol.

2. Description of Related Art

Timolol, as represented by Formula I, is one of the beta-adrenergic antagonists of the thiadiazole compounds, and is also the common name for S-(−)-3-morpholino-4-(3-tert-butylamino-2-hydroxypropoxy)-1,2,5-thiadiazole. The maleic salt of timolol (i.e., timolol maleate) is an antagonist of beta-receptor. It can used to treat glaucoma by reducing aqueous humour production through blockage of the beta receptors on the ciliary epithelium. Timolol is also used to treat hypertension, some cardiovascular diseases and thus prevent heart attack.

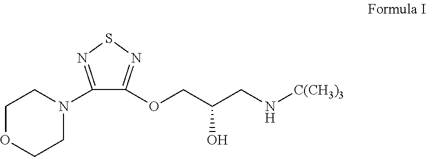

However, it is reported that the R-enantiomer of timolol (i.e., R-(+)-3-morpholino-4-(3-tert-butylamino-2-hydroxypropoxy)-1,2,5-thiadiazole) may be a better choice in treating glaucoma. Several reports suggest that R-enantiomer of timolol can reduce significantly the intraocular pressure in the animal experiment but has a minor side effect when compared with the S-enantiomer of timolol.

Several conventional methods for producing timolol are illustrated as the British patent No. 1253709. One of the synthetic routes is to use 3-hydroxy-4-morpholino-1,2,5-thiadiazole to treat with epichlorohydrin or epibromohydrin to produce the intermediate products comprising 3-morpholino-4-(2,3-epoxypropoxy)-1,2,5-thiadiazole and 3-morpholino-4-(3-chloro-2-hydroxypropoxy)-1,2,5-thiadiazole. The intermediate product then treat with an amine, such as tert-butylamine, to provide the resultant thiadiazole compound 3-morpholino-4-(3-tert-butylamino-2-hydroxypropoxy)-1,2,5-thiadiazole.

Another synthetic route is treat 3-chloro-4-hydroxy-1,2,5-thiadiazole with epichlorohydrin to obtain the first intermediate product 3-chloro-4-(2,3-epoxypropoxy)-1,2,5-thiadiazole. The first intermediate product is then treated with tert-butylamine to obtain the secondary intermediate product 3-chloro-4-(3-tert-butylamino-2-hydroxypropoxy)-1,2,5-thiadiazole. Then the secondary intermediate product is treated with morpholine to obtain the final thiadiazole compound 3-morpholino-4-(3-tert-butylamino-2-hydroxypropoxy)-1,2,5-thiadiazole.

However, the intermediate product and the final thiadiazole compound provided by the abovementioned routes are racemates. In order to obtain an optically active compound, it usually has to use various reagents to isolate one enantiomer from the other via an additional resolution process. As what disclosed in the British patent No. 1253709, the optically active (−)-3-chloro-4-(3-tert-butylamino-2-hydroxypropoxy)-1,2,5-thiadiazole is obtained by treating the racemic 3-chloro-4-(3-tert-butylamino-2-hydroxypropoxy)-1,2,5-thiadiazole, which is the major intermediate product of the producing R-(+)-3-morpholino-4-(3-tert-butylamino-2-hydroxypropoxy)-1,2,5-thiadiazole, with O,O-di-p-toluoyl-(−)-tartaric acid firstly. The (−)-3-chloro-4-(3-tert-butylamino-2-hydroxypropoxy)-1,2,5-thiadiazole is thus separated from its (+)-enantiomer counterpart to the liquid phase, and then obtained by evaporating to dryness, treating with 5N sodium hydroxide, extracting with diethyl ether, washing with water and evaporating to dryness again. Nevertheless, the isolation process is tedious and the yield is usually poor.

There is a need to provide an alternative process for preparation of the optically active timolol, especially in a more convenient and more efficient way than the conventional methods.

### SUMMARY OF THE INVENTION

The present invention provides a process for preparing R-(+)-3-morpholino-4-(3-tert-butylamino-2-hydroxypropoxy)-1,2,5-thiadiazole comprising the following steps.

(a) Reacting a 1,2,5-thiadiazole of formula (1):

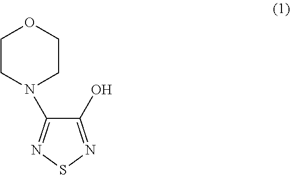

with an S-(+)-epichlorohydrin in the presence of a solvent system which has a first volume and a catalyst optionally in the presence of a suitable base or salt to obtain an optically active intermediate product selected from the group consisting of a first thiadiazole of formula (2)

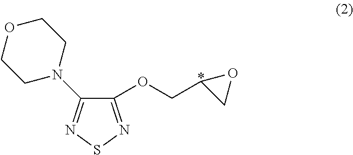

a second thiadiazole of formula (3)

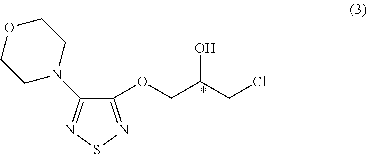

and a mixture of thiadiazoles of formula (2) and formula (3), wherein * means a chiral carbon.

The solvent system can be an amide solvent, sulfoxide solvent, cyclic hydrocarbon solvent, ketone solvent, or a heterocyclic solvent, and the catalyst can be an alkali metal hydroxide, alkali metal carbonate, alkali metal hydrogen carbonate, organic amine, quaternary ammonium cation or other heterocyclic bases.

(b) Treating the mixture of optically active intermediate thiadiazoles obtained from step (a) with a solution, which has a second volume and comprises tert-butylamine to obtain an optically active timolol.

Therefore, one of the objects of the present invention is to provide a process for preparing R-(+)-3-morpholino-4-(3-tert-butylamino-2-hydroxypropoxy)-1,2,5-thiadiazole free of additional resolution steps.

Another object of the present invention is to provide a process for preparing R-(+)-3-morpholino-4-(3-tert-butylamino-2-hydroxypropoxy)-1,2,5-thiadiazole more efficiently and more conveniently.

The present invention also provides a pharmaceutical composition comprising an R-(+)-3-morpholino-4-(3-tert-butylamino-2-hydroxypropoxy)-1,2,5-thiadiazole obtained from the process according to the present invention and a pharmaceutically acceptable carriers, excipient or diluents.

Yet another object of the present invention is to provide a pharmaceutical composition comprising an R-(+)-3-morpholino-4-(3-tert-butylamino-2-hydroxypropoxy)-1,2,5-thiadiazole, wherein the manufacturing process is free of resolution steps.

And, still another object of the present invention is to provide a pharmaceutical composition comprising an R-(+)-3-morpholino-4-(3-tert-butylamino-2-hydroxypropoxy)-1,2,5-thiadiazole more efficiently and more conveniently obtained from the process according to the present invention and a pharmaceutically acceptable carrier, excipient or diluents.

### DETAILED DESCRIPTION OF THE PREFERRED EMBODIMENT

While the present invention discloses a process of preparing optically active timolol, it is to be stated first of all that the detailed organic chemical synthesis or the processing procedures relay on known technology and need not be discussed at length herein.

In one preferred embodiment of the present invention, it discloses a process for preparing R-(+)-3-morpholino-4-(3-tert-butylamino-2-hydroxypropoxy)-1,2,5-thiadiazole. The process comprises the following steps.

(A) Reacting a 3-hydroxy-4-morpholino-1,2,5-thiadiazole of formula (1) with an S-(+)-epichlorohydrin in the presence of a solvent system which has a first volume and a catalyst optionally in the presence of a suitable base to obtain an optically active intermediate product comprising optically active intermediate thiadiazoles of formula (2) and/or formula (3).

(B) Treating the optically active intermediate product obtained from step (A) with a solution that has a second volume and comprises tert-butylamine to obtain an R-(+)-3-morpholino-4-(3-tert-butylamino-2-hydroxypropoxy)-1,2,5-thiadiazole.

### Step (A)

Firstly, 3-hydroxy-4-morpholino-1,2,5-thiadiazole is treated with an (S)-(+)-epichlorohydrin, in the presence of a solvent which has a first volume and a catalyst to get an optically active intermediate product.

In addition, the solvent used in step (A) could be an amide solvent, sulfoxide solvent, cyclic hydrocarbon solvent, ketone solvent, or a heterocyclic solvent. The preferred amide solvent could be N,N-dimethylformamide, dimethylacetamide or mixtures thereof, and the preferred sulfoxide solvent is dimethyl sulfoxide. The preferred cyclic hydrocarbon solvent could be cyclohexane, cycloheptane, benzene, toluene, xylene or mixtures thereof. The preferred ketone solvent is methyl ethyl ketone, acetone or mixtures thereof. The preferred heterocyclic solvent could be tetrahydrofuran, tetrahydropyran, dioxane, or mixtures thereof. The catalyst can be an alkali metal hydroxide, alkali metal carbonate, alkali metal hydrogen carbonate, organic amine, quaternary ammonium cation or other heterocyclic bases. The preferred alkali metal hydroxide is potassium hydroxide or sodium hydroxide and the preferred alkali metal carbonate is potassium carbonate. The preferred organic amine catalyst could be piperidine, pyridine, dicyclohexylamines, triethylamine, or diisopropylethylamine.

The amount of the (S)-(+)-epichlorohydrin is usually used in excess for its solvent properties and the molar ratio of the 1,2,5-thiadiazole to the (S)-(+)-epichlorohydrin preferably ranges from about 1:1 to 1:10. Ideally, the amount of the (S)-(+)-epichlorohydrin ranges from 1 mole to 10 moles, preferably 1.5 mole, when the amount of the 3-hydroxy-4-morpholino-1,2,5-thiadiazole is 1 mole.

The reaction of 3-hydroxy-4-morpholino-1,2,5-thiadiazole and (S)-(+)-epichlorohydrin is carried out, preferably by heating and stirring, in a first temperature for a first period of time, followed by being concentrated in vacuo under a low pressure to obtain an oily residue. The resultant oily residue comprises two optically active intermediate thiadiazoles of formula (2) (i.e., (R)-3-morpholino-4-(2,3-epoxypropoxy)-1,2,5-thiadiazole) and formula (3) (i.e., (S)-3-morpholino-4-(3-chloro-2-hydroxypropoxy)-1,2,5-thiadiazole).

The reaction of step (A) is depicted in Scheme I.

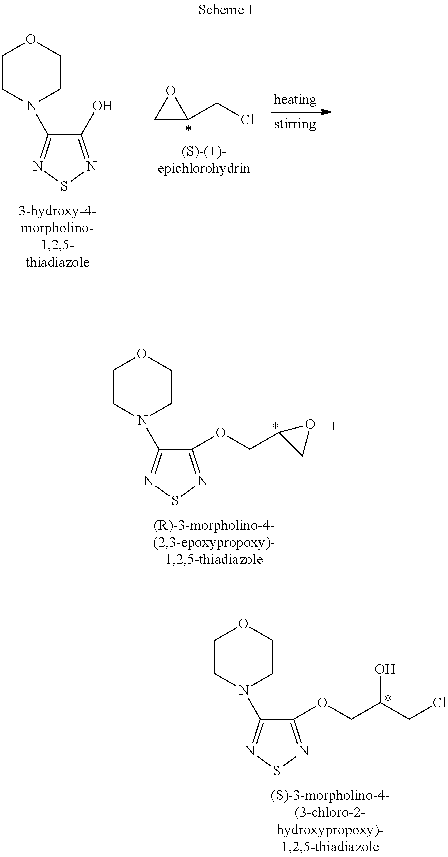

The oily residue comprising the optically active intermediate product obtained from step (A) can be optionally treated with an alkali, such as sodium hydroxide, in an aqueous solution to convert most of the (S)-3-morpholino-4-(3-chloro-2-hydroxypropoxy)-1,2,5-thiadiazole to the (R)-3-morpholino-4-(2,3-epoxypropoxy)-1,2,5-thiadiazole.

The reaction of this optional step is depicted in Scheme Ia.

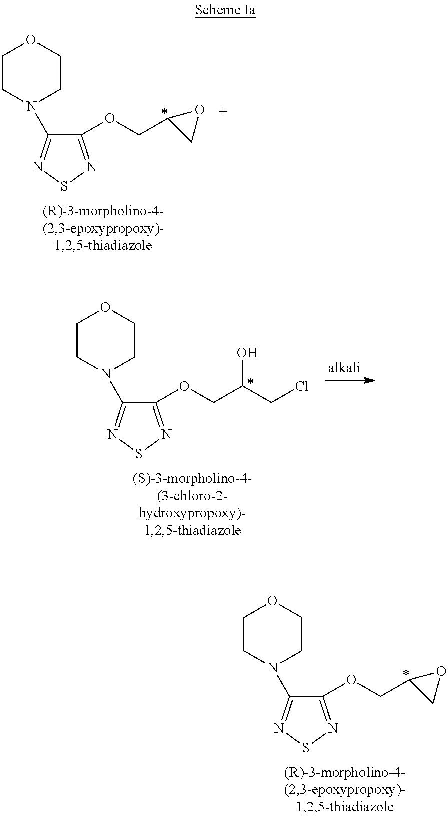

The chiral carbons of every optically active product depicted in Scheme I and Ia are labeled with an astro-mark “*”. The first temperature ranges from about room temperature to about 80° C., preferably ranges from about 60° C. to about 65° C., the first period of time ranges from about 2 hours to about 48 hours.

In addition, the optimal period of time the reaction of step (A) varies from the kinds of solvent and the categories of catalyst used in the reaction. For example, when the solvent is N,N-Dimethylformamide and the catalyst is piperidine, it is preferably heated and stirred for about 8 hours to make 3-hydroxy-4-morpholino-1,2,5-thiadiazole and (S)-(+)-epichlorohydrin to react and then, followed by being concentrated in vacuo, to obtain the oily residue of the optically active intermediate thiadiazoles.

When the solvent is methyl ethyl ketone and the catalyst is potassium hydroxide or sodium hydroxide, it is preferably heated and stirred for about 24 hours to make 3-hydroxy-4-morpholino-1,2,5-thiadiazole and (S)-(+)-epichlorohydrin to react and then, followed by being concentrated in vacuo, to obtain the oily residue of the optically active intermediate thiadiazoles.

When the solvent is tetrahydrofuran and the catalyst is sodium carbonate, or when the solvent is dioxane and the catalyst is piperidine, it is preferably heated and stirred for about 14.5 hours to make the mixture comprising 3-hydroxy-4-morpholino-1,2,5-thiadiazole and (S)-(+)-epichlorohydrin to react and then, followed by being concentrated in vacuo, to obtain the oily residue of the optically active intermediate thiadiazoles.

When the solvent is xylene and the catalyst is potassium hydroxide, it is preferably heated and stirred for a longer period of time, such as about 46 hours, or even longer for 48 hours if the catalyst is potassium carbonate, to make the mixture comprising 3-hydroxy-4-morpholino-1,2,5-thiadiazole and (S)-(+)-epichlorohydrin to react and then, followed by being concentrated in vacuo, to obtain the oily residue of the optically active intermediate thiadiazoles.

### Step (B)

After the optically active intermediate product (i.e., (S)-3-morpholino-4-(3-chloro-2-hydroxypropoxy)-1,2,5-thiadiazole and the (R)-3-morpholino-4-(2,3-epoxypropoxy)-1,2,5-thiadiazole obtained from Scheme I, or (R)-3-morpholino-4-(2,3-epoxypropoxy)-1,2,5-thiadiazole obtained from Scheme Ia) is obtained, it is then treated with a solution comprising tert-butylamine by heating and stirring, for a second temperature for a second period of time to obtain a second mixture. The solution comprising tert-butylamine has a second volume. The solution comprising tert-butylamine can be a pure tert-butylamine, or tert-butylamine in an solvent which is water, ethanol solvent, amide solvent, sulfoxide solvent, cyclic hydrocarbon solvent, ketone solvent, or heterocyclic solvent. The second temperature for reaction of this step ideally ranges from about room temperature to about 70° C., preferably from 30° C. to 50° C., more preferably from 44° C. to 46° C. The second period of time is preferable about 3 hours.

The second mixture is then concentrated in vacuo under a low pressure to obtain an R-(+)-3-morpholino-4-(3-tert-butylamino-2-hydroxypropoxy)-1,2,5-thiadiazole.

The reaction of step (B) is depicted in Scheme II as follows

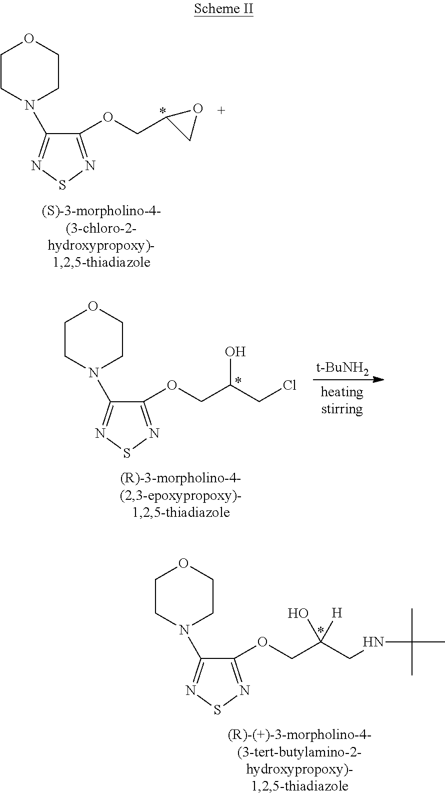

Besides, if the optically active intermediate product is obtained from Scheme Ia (treating with alkali to convert most of the (S)-3-morpholino-4-(3-chloro-2-hydroxypropoxy)-1,2,5-thiadiazole to the (R)-3-morpholino-4-(2,3-epoxypropoxy)-1,2,5-thiadiazole, the reaction of treating with a solution comprising tert-butylamine to obtain an R-(+)-3-morpholino-4-(3-tert-butylamino-2-hydroxypropoxy)-1,2,5-thiadiazole is depicted as Scheme IIa.

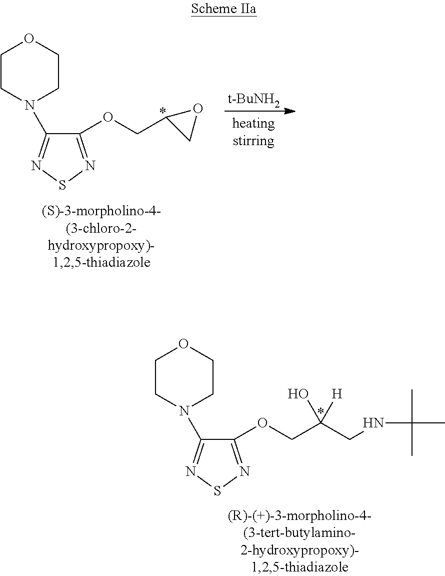

The chiral carbons of every optically active compound are also labeled with an astro-mark “*”. Besides, the ideal volumetric ratio of the solvent (i.e., the first volume) used in step (A) to the solution comprising tert-butylamine (i.e., the second volume) is about 1:18.

In addition, the R-(+)-3-morpholino-4-(3-tert-butylamino-2-hydroxypropoxy)-1,2,5-thiadiazole obtained from the process disclosed according to the present invention is substantially optically pure, that is, having a high enantiomeric excess (also abbreviated as “e.e.”) value. High e.e. value usually represent the success of the synthetic process concerning about the asymmetric synthesis. After analysis, the e.e. value of the R-(+)-3-morpholino-4-(3-tert-butylamino-2-hydroxypropoxy)-1,2,5-thiadiazole obtained from step (B) is at least 95%, if S-(+)-epichlorohydrin is used in step (A).

### Step (C)

Optionally, after step (B), the process according to the present invention could further comprise a step (C) to treat the R-(+)-3-morpholino-4-(3-tert-butylamino-2-hydroxypropoxy)-1,2,5-thiadiazole free base obtained from step (B) with maleic acid followed by being stirred, filtrated, and recrystallized to obtain an R-(+)-3-morpholino-4-(3-tert-butylamino-2-hydroxypropoxy)-1,2,5-thiadiazole hydrogen maleate. This step is depicted in Scheme III as follows.

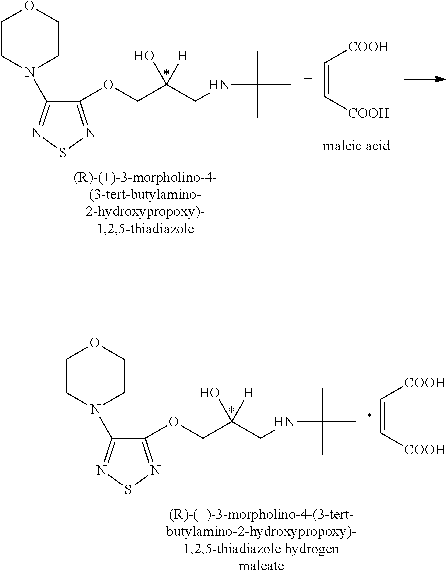

The R-(+)-3-morpholino-4-(3-tert-butylamino-2-hydroxypropoxy)-1,2,5-thiadiazole hydrogen maleate obtained from the step (C), after analysis, still has a high e.e. value which is at least 98%, and the yield of the R-(+)-3-morpholino-4-(3-tert-butylamino-2-hydroxypropoxy)-1,2,5-thiadiazole hydrogen maleate after step (C) is usually at least 88%.

Another preferred embodiment of the present invention provides a pharmaceutical composition comprising an optically active timolol prepared according to the process as described above and a pharmaceutically acceptable carrier, excipient or diluents.

The preferable pharmaceutically acceptable diluents include but are not limited to sorbitol, mannitol, starch, lactose, cellulose in powder form or microcrystalline form, dicalcium phosphate, tricalcium phosphates, sugar and the like. Other pharmaceutically acceptable carriers and excipients include but are not limited to binders, disintegrants, lubricants, glidants, solubility or wet enhancers, complex forming agents, release controlling agents, film formers, plasticizers, colorants, flavoring agents, sweeteners, viscosity enhancers, preservatives, antioxidants and the like.

Several specific examples of this invention are described in details and as below, and are provided only for purpose of illustration and are not intended to limit the scope of the invention as disclosed in the claims.

### Example 1

### Preparation of R-(+)-3-morpholino-4-(3-tert-butylamino-2-hydroxypropoxy)-1,2,5-thiadiazole

30.0 grams of 3-hydroxy-4-morpholino-1,2,5-thiadiazole and 21.8 ml of S-(+)-epichlorohydrin is mixed in the present of 80.0 ml of methyl ethyl ketone and 2.4 grams of sodium hydroxide. The mixture is heated to and maintained at 60° C.˜65° C., and, in the meantime, stirred for 24 hours. Excess S-(+)-epichlorohydrin is removed by concentration in vacuo under a low pressure about 750 mmHg and at the temperature about 80° C. The oily residue is then obtained. The obtained oily residue is mixed with 400.0 ml of tert-butylamine, followed by being heating to and maintaining at 44° C.˜46° C. and, in the meantime, being stirring for 3 hours. Excess tert-butylamine is removed by vacuum and concentrating under a low pressure about 720 mmHg and at the temperature about 40° C. The obtained oily residue is R-(+)-3-morpholino-4-(3-tert-butylamino-2-hydroxypropoxy)-1,2,5-thiadiazole. The purity of the final product obtained is 98.1% in R-(+)-form and 1.9% in S-(−)-form by HPLC. Thus, the e.e. value of the R-(+)-3-morpholino-4-(3-tert-butylamino-2-hydroxypropoxy)-1,2,5-thiadiazole obtained from this preparation is 96.2%.

### Example 2

### Preparation of R-(+)-3-morpholino-4-(3-tert-butylamino-2-hydroxypropoxy)-1,2,5-thiadiazole

30.0 grams of 3-hydroxy-4-morpholino-1,2,5-thiadiazole and 21.8 ml of S-(+)-epichlorohydrin is mixed in the present of 80.0 ml of methyl ethyl ketone and 3.4 grams of potassium hydroxide. The mixture is heated to and maintained at 60° C.˜65° C., and, in the meantime, stirred for 14 hours Excess S-(+)-epichlorohydrin is removed by concentration in vacuo under a low pressure about 750 mmHg and at the temperature about 80° C. The oily residue is then obtained. The obtained oily residue is mixed with 400.0 ml of tert-butylamine, followed by being heating to and maintaining at 44° C.˜46° C. and, in the meantime, being stirring for 3 hours. Excess tert-butylamine is removed by concentration in vacuo under a low pressure about 720 mmHg and at the temperature about 40° C. The obtained oily residue is R-(+)-3-morpholino-4-(3-tert-butylamino-2-hydroxypropoxy)-1,2,5-thiadiazole. The purity of the final product obtained is 99.1% in R-(+)-form and 0.9% in S-(−)-form by HPLC. Thus, the e.e. value of the R-(+)-3-morpholino-4-(3-tert-butylamino-2-hydroxypropoxy)-1,2,5-thiadiazole obtained from this preparation is 98.2%.

### Example 3

### Preparation of R-(+)-3-morpholino-4-(3-tert-butylamino-2-hydroxypropoxy)-1,2,5-thiadiazole

30.0 grams of 3-hydroxy-4-morpholino-1,2,5-thiadiazole and 21.8 ml of S-(+)-epichlorohydrin is mixed in the present of 80.0 ml of xylene and 3.4 grams of potassium hydroxide. The mixture is heated to and maintained at 60° C.˜65° C., and, in the meantime, stirred for 46 hours. Excess S-(+)-epichlorohydrin is removed by concentration in vacuo under a low pressure about 750 mmHg and at the temperature about 80° C. The oily residue is then obtained. The obtained oily residue is mixed with 400.0 ml of tert-butylamine, followed by being heating to and maintaining at 44° C.˜46° C. and, in the meantime, being stirring for 3 hours. Excess tert-butylamine is removed by concentration in vacuo under a low pressure about 720 mmHg and at the temperature about 40° C. The obtained oily residue is R-(+)-3-morpholino-4-(3-tert-butylamino-2-hydroxypropoxy)-1,2,5-thiadiazole. The purity of the final product obtained is 98.5% in R-(+)-form and 1.5% in S-(−)-form by HPLC. Thus, the e.e. value of the R-(+)-3-morpholino-4-(3-tert-butylamino-2-hydroxypropoxy)-1,2,5-thiadiazole obtained from this preparation is 97.0%.

### Example 4

### Preparation of R-(+)-3-morpholino-4-(3-tert-butylamino-2-hydroxypropoxy)-1,2,5-thiadiazole

30.0 grams of 3-hydroxy-4-morpholino-1,2,5-thiadiazole and 21.8 ml of S-(+)-epichlorohydrin is mixed in the present of 80.0 ml of tetrahydrofuran and 6.4 grams of sodium carbonate. The mixture is heated to and maintained at 60° C.˜65° C., and, in the meantime, stirred for 14.5 hours. Excess S-(+)-epichlorohydrin is removed by concentration in vacuo under a low pressure about 750 mmHg and at the temperature about 80° C. The oily residue is then obtained. The obtained oily residue is mixed with 400.0 ml of tert-butylamine, followed by being heating to and maintaining at 44° C.˜46° C. and, in the meantime, being stirring for 3 hours. Excess tert-butylamine is removed by concentration in vacuo under a low pressure about 720 mmHg and at the temperature about 40° C. The obtained oily residue is R-(+)-3-morpholino-4-(3-tert-butylamino-2-hydroxypropoxy)-1,2,5-thiadiazole. The purity of the final product obtained is 99.3% in R-(+)-form and 0.7% in S-(−)-form by HPLC. Thus, the e.e. value of the R-(+)-3-morpholino-4-(3-tert-butylamino-2-hydroxypropoxy)-1,2,5-thiadiazole obtained from this preparation is 98.6%.

### Example 5

### Preparation of R-(+)-3-morpholino-4-(3-tert-butylamino-2-hydroxypropoxy)-1,2,5-thiadiazole

30.0 grams of 3-hydroxy-4-morpholino-1,2,5-thiadiazole and 21.8 ml of S-(+)-epichlorohydrin is mixed in the present of 80.0 ml of dioxane and 5.1 grams of piperidine. The mixture is heated to and maintained at 60° C.˜65° C., and, in the meantime, stirred for 14.5 hours. Excess S-(+)-epichlorohydrin is removed by concentration in vacuo under a low pressure about 750 mmHg and at the temperature about 80° C. The oily residue is then obtained. The obtained oily residue is mixed with 400.0 ml of tert-butylamine, followed by being heating to and maintaining at 44° C.˜46° C. and, in the meantime, being stirring for 3 hours. Excess tert-butylamine is removed concentration in vacuo under a low pressure about 720 mmHg and at the temperature about 40° C. The obtained oily residue is R-(+)-3-morpholino-4-(3-tert-butylamino-2-hydroxypropoxy)-1,2,5-thiadiazole. The purity of the final product obtained is 99.0% in R-(+)-form and 1.0% in S-(−)-form by HPLC. Thus, the e.e. value of the R-(+)-3-morpholino-4-(3-tert-butylamino-2-hydroxypropoxy)-1,2,5-thiadiazole obtained from this preparation is 98.0%.

### Example 6

### Preparation of R-(+)-3-morpholino-4-(3-tert-butylamino-2-hydroxypropoxy)-1,2,5-thiadiazole

30.0 grams of 3-hydroxy-4-morpholino-1,2,5-thiadiazole and 21.8 ml of S-(+)-epichlorohydrin is mixed in the present of 80.0 ml of N,N-dimethylformamide and 5.1 grams of piperidine. The mixture is heated to and maintained at 60° C.˜65° C., and, in the meantime, stirred for 8 hours. Excess S-(+)-epichlorohydrin is removed by concentration in vacuo under a low pressure about 750 mmHg and at the temperature about 80° C. The oily residue is then obtained. The obtained oily residue is mixed with 400.0 ml of tert-butylamine, followed by being heating to and maintaining at 44° C.˜46° C. and, in the meantime, being stirring for 3 hours. Excess tert-butylamine is removed by concentration in vacuo under a low pressure about 720 mmHg and at the temperature about 40° C. The obtained oily residue is R-(+)-3-morpholino-4-(3-tert-butylamino-2-hydroxypropoxy)-1,2,5-thiadiazole. The purity of the final product obtained is 98.6% in R-(+)-form and 1.4% in S-(−)-form by HPLC. Thus, the e.e. value of the R-(+)-3-morpholino-4-(3-tert-butylamino-2-hydroxypropoxy)-1,2,5-thiadiazole obtained from this preparation is 97.2%.

### Example 7

### Preparation of R-(+)-3-morpholino-4-(3-tert-butylamino-2-hydroxypropoxy)-1,2,5-thiadiazole

30.0 grams of 3-hydroxy-4-morpholino-1,2,5-thiadiazole and 21.8 ml of S-(+)-epichlorohydrin is mixed in the present of 80.0 ml of xylene and 8.3 grams of potassium carbonate. The mixture is heated to and maintained at 60° C.˜65° C., and, in the meantime, stirred for 48 hours. Excess S-(+)-epichlorohydrin is removed by concentration in vacuo under a low pressure about 750 mmHg and at the temperature about 80° C. The oily residue is then obtained. The obtained oily residue is mixed with 400.0 ml of tert-butylamine, followed by being heating to and maintaining at 44° C.˜46° C. and, in the meantime, being stirring for 3 hours. Excess tert-butylamine is removed by concentration in vacuo under a low pressure about 720 mmHg and at the temperature about 40° C. The obtained oily residue is R-(+)-3-morpholino-4-(3-tert-butylamino-2-hydroxypropoxy)-1,2,5-thiadiazole. The purity of the final product obtained is 97.9% in R-(+)-form and 2.1% in S-(−)-form by HPLC. Thus, the e.e. value of the R-(+)-3-morpholino-4-(3-tert-butylamino-2-hydroxypropoxy)-1,2,5-thiadiazole obtained from this preparation is 95.8%.

### Example 8

### Preparation of R-(+)-3-morpholino-4-(3-tert-butylamino-2-hydroxypropoxy)-1,2,5-thiadiazole hydrogen maleate

10.0 grams of R-(+)-3-morpholino-4-(3-tert-butylamino-2-hydroxypropoxy)-1,2,5-thiadiazole obtained from Example 1 is dissolved in 30.0 ml of isopropanol, and 3.7 grams of maleic acid is then mixed with the resultant solution, followed by stirring at room temperature for an hour. 12.3 grams of R-(+)-3-morpholino-4-(3-tert-butylamino-2-hydroxypropoxy)-1,2,5-thiadiazole hydrogen maleate crude product was then obtained after filtration. The crude product is then recrystallized in the presence of 37.0 ml of ethanol, and 9.8 grams of fine product is thus obtained. The purity of the final product obtained is 99.67% in R-(+)-form and 0.33% in S-(−)-form by HPLC. Thus, the percent e.e. value of the R-(+)-3-morpholino-4-(3-tert-butylamino-2-hydroxypropoxy)-1,2,5-thiadiazole hydrogen maleate obtained from this preparation is 99.34%.

Although the particular embodiments of the invention have been described in detail for purpose of illustration, it will be understood by one of ordinary skill in the art that numerous variations will be possible to the disclosed embodiments without going outside the scope of the invention as disclosed in the claims.

What is claimed is:

1. A process for preparing R-(+)-3-morpholino-4-(3-tert-butylamino-2-hydroxypropoxy)-1,2,5-thiadiazole comprising the steps of:
(a) reacting a 1,2,5-thiadiazole of formula (1): 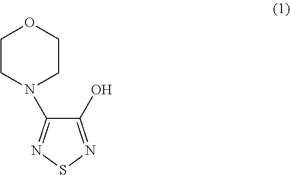
with an S-(+)-epichlorohydrin in the presence of a solvent system which has a first volume and is selected from the group consisting of amide solvent, sulfoxide solvent, cyclic hydrocarbon solvent, ketone solvent, and heterocyclic solvent, and a catalyst optionally in the presence of a suitable base to obtain an optically active intermediate product selected from the group consisting of a first thiadiazole of formula (2), 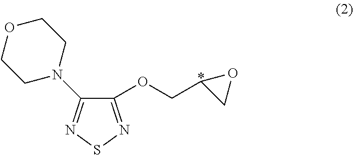
a second thiadiazole of formula (3), 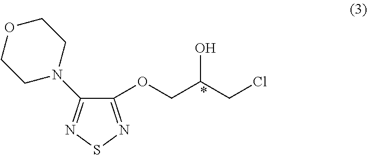
and a mixture thereof,
wherein * means a chiral carbon and said catalyst is selected from the group consisting of alkali metal hydroxide, alkali metal carbonate, alkali metal hydrogen carbonate, organic amine, quaternary ammonium cation and other heterocyclic bases; and
(b) treating said optically active intermediate product obtained from step (a) with a solution which has a second volume and comprises tert-butylamine to obtain an optically active timolol.

2. The process of claim 1, wherein the step (a) further comprises a step of treating said optically active intermediate product with an alkali.

3. The process of claim 1, wherein the solution used in step (b) further comprises a solvent selected from the group consisting of H₂O, ethanol solvent, amide solvent, sulfoxide solvent, cyclic hydrocarbon solvent, ketone solvent, and heterocyclic solvent.

4. The process of claim 1, wherein said amide solvent is selected from the group consisting of N,N-dimethylformamide, dimethylacetamide and mixtures thereof.

5. The process of claim 1, wherein said sulfoxide solvent is dimethyl sulfoxide.

6. The process of claim 1, wherein said cyclic hydrocarbon solvent system is selected from the group consisting of cyclohexane, cycloheptane, benzene, toluene, and xylene and mixtures thereof.

7. The process of claim 1, wherein said ketone solvent is selected from the group consisting of methyl ethyl ketone, acetone, and mixtures thereof.

8. The process of claim 1, wherein said heterocyclic solvent is selected from the group consisting of tetrahydrofuran, tetrahydropyran, dioxane, and mixtures thereof.

9. The process of claim 1, wherein said organic amine is selected from the group consisting of piperidine, pyridine, dicyclohexylamines, triethylamine, and diisopropylethylamine.

10. The process of claim 1, wherein reacting said 1,2,5-thiadiazole of formula (1) with said S-(+)-epichlorohydrin is carried out at a temperature of about room temperature to about 80° C.

11. The process of claim 1, wherein reacting said 1,2,5-thiadiazole of formula (1) with said S-(+)-epichlorohydrin is carried out for a period of time of about 2 hours to about 48 hours.

12. The process of claim 1, wherein treating said first optically active intermediate thiadiazole and said second optically active intermediate thiadiazole with tert-butylamine is carried out at a temperature of about room temperature to about 70° C.

13. The process of claim 1, wherein treating said first optically active intermediate thiadiazole and said second optically active intermediate thiadiazole with tert-butylamine is carried out for a period of time of about 3 hours.

14. The process of claim 1, wherein the volumetric ratio of said first volume to said second volume is about 1:18.

15. The process of claim 1, wherein the molar ratio of said 1,2,5-thiadiazole of formula (1) to said S-(+)-epichlorohydrin ranges from about 1:1 to 1:10.

16. The process of claim 1, further comprising a step of treating said R-(+)-3-morpholino-4-(3-tert-butylamino-2-hydroxypropoxy)-1,2,5-thiadiazole with maleic acid followed by being stirred, filtrated, and recrystallized to obtain an R-(+)-3-morpholino-4-(3-tert-butylamino-2-hydroxypropoxy)-1,2,5-thiadiazole hydrogen maleate.

17. The process of claim 1, said R-(+)-3-morpholino-4-(3-tert-butylamino-2-hydroxypropoxy)-1,2,5-thiadiazole has an optical purity which is at least about 95%.

18. A pharmaceutical composition comprising an R-(+)-3-morpholino-4-(3-tert-butylamino-2-hydroxypropoxy)-1,2,5-thiadiazole obtained from claim 1 and a pharmaceutically acceptable carriers, excipient or diluents.
